(19) United States
(12) Patent Application Publication
(10) Pub. No.: US 2008/0233007 A1
(43) Pub. Date: Sep. 25, 2008

(54) Nondestructive Portable Sugar Level Measurement Instrument with Applications in Food Production and Distribution Industries

(75) Inventors: Awad Al-Khalaf, Sharjah (AE); Maamar Bettayeb, Sharjah (AE); Anas Youssef Boubas, Al Ain (AE)

Correspondence Address: Dr Awad Al-Khalaf, P.O.Box 27272, University of Sharjah, Sharjah, omitted

(73) Assignees: Awad Al-Khalaf, Sharjah (AE); Maamar Bettayeb, Sharjah (AE); Anas Youssef Boubas, Al Ain (AE)

(21) Appl. No.: 11/953,051

(22) Filed: Dec. 9, 2007

Related U.S. Application Data
(60) Provisional application No. 60/896,091, filed on Mar. 21, 2007.

(51) Int. Cl. G01N 33/02

(52) U.S. Cl. 422/68.1

(57) ABSTRACT

We present a device for measuring levels and concentrations of sugar in objects that is suitable for food production and distribution industries. One of the key characteristics of the device is its ability to perform the measurement without destructing the product, and therefore, maintaining its quantity and quality.

The technologies empowering the invention are the nondestructive ultrasonic sensing and the intelligent analysis of data collected. Ultrasonic waves are emitted towards the object from different directions, and the different reflections are collected and passed by for analysis.

The analytic component analyses the signals, and with the use of advanced signal processing technologies, the signatures that represent the presence of sugar are extracted. These, combined with the measurement of the analyzed volume are used to find out the concentration and amount of sugar in the product.

We emphasize on the portability and cost-effectiveness of the invention, hence we use the appropriate technologies that serves best our purpose.

### BRIEF SUMMARY OF THE INVENTION

We present a device for measuring levels and concentrations of sugar in objects that is suitable for food production and distribution industries. One of the key characteristics of the device is its ability to perform the measurement without destructing the product, and therefore, maintaining its quantity and quality.

The technologies empowering the invention are the nondestructive ultrasonic sensing and the intelligent analysis of data collected. Ultrasonic waves are emitted towards the object from different directions, and the different reflections are collected and passed by for analysis.

The analytic component analyses the signals, and with the use of advanced signal processing technologies, the signatures that represent the presence of sugar are extracted. These, combined with the measurement of the analyzed volume are used to find out the concentration and amount of sugar in the product.

We emphasize on the portability and cost-effectiveness of the invention, hence we use the appropriate technologies that serves best our purpose.

### BRIEF DESCRIPTION OF THE VIEW OF THE DRAWING

The invention will now be described by way of example with reference to the accompanying drawing.

FIG. 1: Shows the sensory system with an object placed inside.

### DETAILED DESCRIPTION OF THE INVENTION

The device consists of two major systems, which are the sensory system and the intelligent computing system.

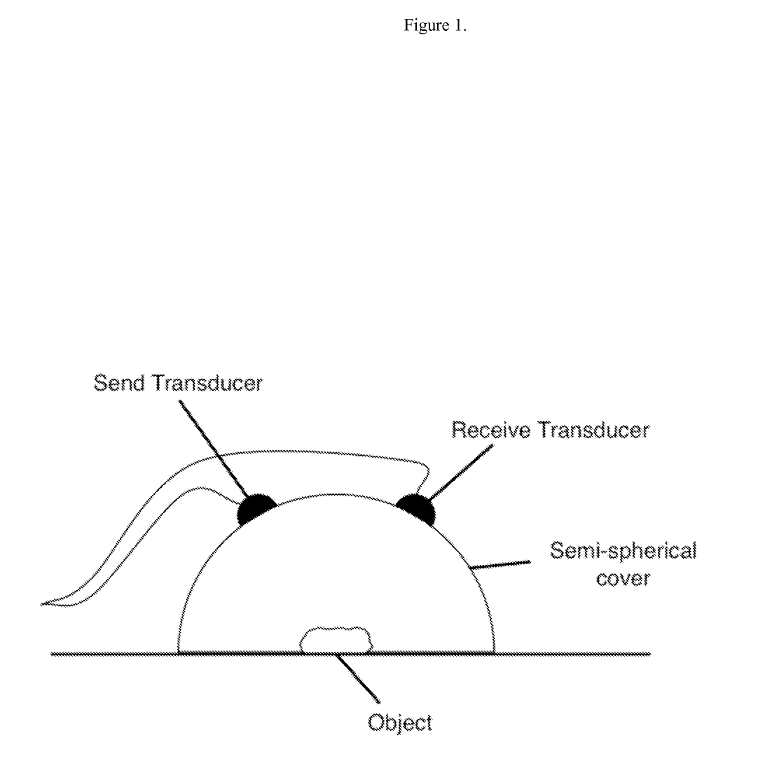

The sensory system performs two major functionalities: measurement of the object's volume, or its cross-section volume, and the measurement of the amount of sugar in that measured volume. The sensory system consists mainly of the following four components: send transducer, receive transducer, movable grating plane and object stand. The two transducers are placed at an angle of θ°, and hence our angle of incidence is taken to be θ°. The sensory system is described in FIG. 1. The transducers can move in such a way that θ° changes, yet the reflections can still be captured by the receive transducer. The transducers can move by shifting on the semi-spherical cover shown in FIG. 1.

Measurement of the volume can be done by the calculation of the time delay between sending and the first receiving of the reflections. Measurement of the amount of sugar in the measured volume will be described below.

Successive ultrasonic tonebursts are emitted by the send transducer at different frequencies with a time delay. Each time an ultrasonic toneburst is emitted at a certain frequency, its reflections are collected by the receive transducer. The reflected intensities relate to the ultrasonic properties of the object. Using the fact that variation in sugar concentrations corresponds to a special kind of variations in the ultrasonic properties of the object, hence the sugar concentration can be estimated using the measurements of reflections.

The intelligent computing system uses the measured volume and amount of sugar to determine the concentration. The intelligent computing system is “intelligent” in the sense that it uses other detections done by the sensory system to predict what class does the object belongs to. Determining the class of the object helps to take into consideration the effect of other substances that normally are contained in that class and interfere with the sugar's ultrasonic signature.

What is claimed is:

1. A sugar level measurement system that determines the sugar level of food products. The sugar level measurement system uses reflections' signature of ultrasonic emissions sent to the food specimen to determine the sugar level in the food specimen. The claimed sugar level measurement system comprises:
a housing of a size suitable to be placed in a pocket;
a micro-computing device mounted within the housing;
an output device that displays results of the micro-computing device;
ultrasonic transducers mounted at suitable angles to send ultrasonic emissions to the specimen of which the sugar level is to be determined, and receive ultrasonic reflections from the specimen of which the sugar level is to be determined;
an input device that allows the user of the sugar level measurement system to activate the sugar level measurement system; and
an input device that allows the user of the sugar level measurement system to order the sugar level measurement system to execute the sugar measurement process.
